(19) United States
(12) Patent Application Publication
(10) Pub. No.: US 2020/0107736 A1
(43) Pub. Date: Apr. 9, 2020

(54) PULSE WAVE MEASUREMENT APPARATUS

(71) Applicant: FUJITSU CONNECTED TECHNOLOGIES LIMITED, Kawasaki-shi (JP)

(72) Inventor: Manabu Ishida, Kawasaki (JP)

(73) Assignee: FUJITSU CONNECTED TECHNOLOGIES LIMITED, Kawasaki-shi (JP)

(21) Appl. No.: 16/394,372

(22) Filed: Apr. 25, 2019

(30) Foreign Application Priority Data
Oct. 9, 2018 (JP) 2018-191168

(51) Int. Cl. A61B 5/024; A61B 5/00

(52) U.S. Cl. CPC A61B 5/02427; A61B 5/02433; A61B 5/6826

(57) ABSTRACT

A pulse wave measurement apparatus includes a light transmitting plate, a green light which casts green light, an infrared light which casts infrared light, a memory, and a processor coupled to the memory. The processor is configured to cause the green light to cast green light on the finger through the light transmitting plate, the green light having a peak wavelength in a wavelength range of 490 nm or more and 570 nm or less, detect an intensity of reflection of the casted green light from the finger, measure a pulse wave in the finger by making the infrared light cast infrared light on the finger through the light transmitting plate when the intensity of the reflection indicates that force at which the finger is pressed against the light transmitting plate is within a predetermined range suitable for the measurement of the pulse wave.

### CROSS-REFERENCE TO RELATED APPLICATION

This application is based upon and claims the benefit of priority of the prior Japanese Patent Application No. 2018-191168, filed on Oct. 9, 2018, the entire contents of which are incorporated herein by reference.

### FIELD

The embodiments discussed herein are related to a pulse wave measurement apparatus.

### BACKGROUND

A pulse wave detection apparatus is used which casts infrared light on a finger pressed against the apparatus and measures a pulse wave based on reflection from the finger.

[Patent document 1] Japanese Laid-open Patent Publication No. 2016-36411

### SUMMARY

A pulse wave measurement apparatus includes a light transmitting plate which is made of a member capable of transmitting light and against which a finger is pressed, a green light which casts green light, an infrared light which casts infrared light, a memory; and a processor coupled to the memory. The processor is configured to cause the green light to cast green light on the finger through the light transmitting plate, the green light having a peak wavelength in a wavelength range of 490 nm or more and 570 nm or less, detect an intensity of reflection of the casted green light from the finger, measure a pulse wave in the finger by making the infrared light cast infrared light on the finger through the light transmitting plate when the intensity of the reflection indicates that force at which the finger is pressed against the light transmitting plate is within a predetermined range suitable for the measurement of the pulse wave.

The object and advantages of the invention will be realized and attained by means of the elements and combinations particularly pointed out in the claims. It is to be understood that both the foregoing general description and the following detailed description are exemplary and explanatory and are not restrictive of the invention.

### BRIEF DESCRIPTION OF DRAWINGS

FIG. 1 is a view illustrating an example of a pulse wave measurement apparatus according to a first embodiment;

FIGS. 2A and 2B are views schematically illustrating relationships between the wavelength of light casted on the finger and depth which the casted light reaches in the finger;

FIG. 3 is a view illustrating an example of correspondence among the magnitude of force at which the finger is pressed against a light transmitting plate, the intensity of reflection of green light received by a light receiver, and a measured pulse wave in the first embodiment;

FIG. 4 is an example of a timing chart of the pulse wave measurement apparatus according to the first embodiment;

FIG. 5 is a view illustrating an example of a processing flow of the pulse wave measurement apparatus according to the first embodiment;

FIG. 6 is a view illustrating an example of a pulse wave measurement apparatus according to a comparative example; and

FIG. 7 is a view illustrating an example of correspondence among the magnitude of the force at which the finger is pressed against the light transmitting plate, the degree of redness in the color of the finger captured by a camera sensor, and the measured pulse wave in the comparative example.

### DESCRIPTION OF EMBODIMENTS

When the finger is not pressed at suitable force, the detection accuracy of the pulse wave decreases. Accordingly, the pulse wave detection apparatus determines whether the fingertip is pressed at the suitable force or not before the measurement of the pulse wave. Such determination is performed based on the color of an image of the fingertip captured while casting white light on the pressed finger.

As a result of earnest studies, the present inventor found that such a technique has the following problems. First, it was found that since the casted white light reaches the artery in the finger, the captured image tends to be reddish. As a result, it is difficult for the pulse wave detection apparatus to distinguish between the case where the red image is obtained due to strong pressing of the finger and the case where the red image is obtained as a result of the white light reaching the artery, though the finger is pressed at the suitable force. Moreover, the change in the color tone of the fingertip of a person with dark skin color is smaller than that of a person with light skin color. Accordingly, in the case of dark skin color, it is difficult for the pulse wave detection apparatus to determine whether the finger is pressed at the suitable force or not.

An object of one aspect of the disclosed technique is to provide a pulse wave measurement apparatus which can more accurately detect that the finger is pressed at suitable force.

An embodiment is described below. A configuration of the embodiment described below is an example and the disclosed technique is not limited to the configuration of the embodiment. A pulse wave measurement apparatus according to the embodiment has, for example, the following configuration.

(Configuration of Pulse Wave Measurement Apparatus)

A pulse wave measurement apparatus includes a light transmitting plate which is made of a member capable of transmitting light and against which a finger is pressed, a green light which casts green light, an infrared light which casts infrared light, a memory; and a processor coupled to the memory. The processor is configured to cause the green light to cast green light on the finger through the light transmitting plate, the green light having a peak wavelength in a wavelength range of 490 nm or more and 570 nm or less, detect an intensity of reflection of the casted green light from the finger, measure a pulse wave in the finger by making the infrared light cast infrared light on the finger through the light transmitting plate when the intensity of the reflection indicates that force at which the finger is pressed against the light transmitting plate is within a predetermined range suitable for the measurement of the pulse wave.

The pulse wave measurement apparatus measures the pulse wave by casting light on the finger pressed against the light transmitting plate. The pulse wave measurement apparatus determines whether the finger is pressed at force suitable for the measurement of the pulse wave or not, based on the intensity of reflection of green light emitted by the light casting unit. The green light emitted by the light casting unit has a peak wavelength in a wavelength range of 490 nm or more and 570 nm or less. When the green light having the peak wavelength in such a wavelength band is casted on the finger, the casted green light reaches the capillaries inside the finger but hardly reaches the artery and the vein located deeper than the capillaries. Accordingly, the amount of the green light absorbed by the blood out of the green light casted on the finger, that is the intensity of the light reflected on the finger and detected by the detector changes depending on a flow rate of blood flowing in the capillaries.

When the finger is pressed against the light transmitting plate at high force, the capillaries are crushed and the flow rate of the blood flowing in the capillaries decreases. When the finger is pressed against the light transmitting plate at low force, the capillaries are not crushed. Thus, the flow rate of the blood flowing in the capillaries is greater than that in the case where the finger is pressed at the high force. The blood flow rate changes substantially linearly depending on the force at which the finger is pressed. Accordingly, in the embodiment, whether the finger is pressed with suitable force or not can be more accurately determined by detecting the change in the flow rate of blood in the capillaries depending on the force at which the finger is pressed against the light transmitting plate, as the intensity of reflection.

The embodiment may have the following characteristics. The detector may have a characteristic of transitioning to a state in which the detector can detect the intensity of the reflection in at least part of a period from the moment when the green light is emitted to a moment when the infrared light is emitted. The pulse wave measurement apparatus having such a characteristic can achieve lower power consumption than in the case where the detector is kept operating.

The disclosed technique may have the following characteristic. When the intensity of the reflection indicates that the force is outside the predetermined range, the execution unit cancels the casting of the infrared light by the measurement unit. The pulse wave measurement apparatus having such a characteristic can cancel the execution of the measurement of the pulse wave in a situation not suitable for the measurement of the pulse wave.

The embodiment may have the following characteristic. When the intensity of the reflection indicates that the force is outside the predetermined range, the execution unit notifies a user that the force at which the finger is pressed against the light transmitting plate is outside the predetermined range. The pulse wave measurement apparatus having such a characteristic can prompt the user to adjust the force at which the finger is pressed.

The embodiment is further described below with reference to the drawings.

### First Embodiment

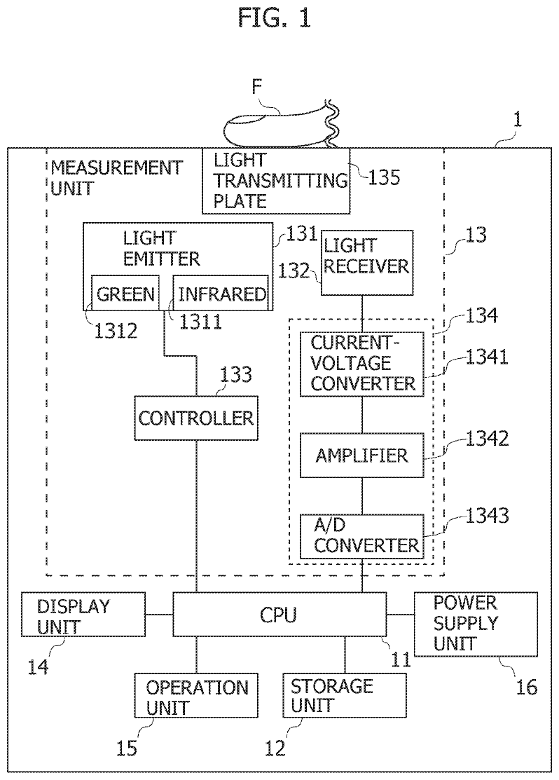

FIG. 1 is a view illustrating an example of a pulse wave measurement apparatus according to a first embodiment. The pulse wave measurement apparatus 1 according to the first embodiment is an apparatus which measures a pulse wave by casting light on the pressed fingertip. The pulse wave measurement apparatus 1 includes a Central Processing Unit (CPU) 11, a storage unit 12, a measurement unit 13, a display unit 14, an operation unit 15, and a power supply unit 16. In FIG. 1, the finger F which is a pulse wave measurement target is also illustrated as an example.

The measurement unit 13 includes a light emitter 131, a light receiver 132, a controller 133, a converter 134, and a light transmitting plate 135. The light transmitting plate 135 is a member against which the ball (surface opposite to the nail in the fingertip) of the finger F being the pulse wave measurement target is pressed and is made of a member which transmits light. The light transmitting plate 135 is an example of the “light transmitting plate.”

The controller 133 causes the light emitter 131 to emit either the green light or the infrared light in response to an instruction from the CPU 11. The light emitter 131 includes an infrared light emitter 1311 configured to emit the infrared light and a green light emitter 1312 configured to emit the green light having a shorter wavelength than the infrared light. The light emitter 131 emits the green light or the infrared light in response to an instruction from the controller 133. The light emitted from the light emitter 131 is casted on the finger pressed against the light transmitting plate 135. Part of the light casted on the finger is absorbed by the blood flowing in the blood vessel in the finger. Part of the light casted on the finger and not absorbed by the blood is reflected and the reflection is received by the light receiver 132.

The green light emitter 1312 emits, for example, green light with the peak wavelength within the range of 490 nm to 570 nm. The green light emitted by light emission of the green light emitter 1312 passes through the light transmitting plate 135 and is casted on the finger F pressed against the light transmitting plate 135. The green light emitter 1312 is, for example, a Light Emitting Diode (LED) configured to emit the green light. The infrared light emitter 1311 emits the infrared light. The infrared light emitted by the light emission of the infrared light emitter 1311 passes through the light transmitting plate 135 and is casted on the finger F pressed against the light transmitting plate 135. The infrared light emitter 1311 is, for example, an LED configured to emit the infrared light. The light emitter 131 is an example of the “light casting unit.”

The light receiver 132 receives the reflection of the green light or the infrared light emitted from the light emitter 131 and reflected on the finger F pressed against the light transmitting plate 135. The light receiver 132 converts the received light to an electric current and outputs the electric current obtained by the conversion to the converter 134. The light receiver 132 is switched between a first state in which the light receiver 132 can receive the reflection of the green light and output the electric current and a second state in which the light receiver 132 can receive the reflection of the infrared light and output the electric current, in a time division manner. Specifically, in a period of the first state, the light receiver 132 outputs the electric current depending on the intensity of the received reflection of the green light but does not output the electric current even if the light receiver 132 receives the reflection of the infrared light. Moreover, in a period of the second state, the light receiver 132 outputs the electric current depending on the intensity of the received reflection of the infrared light but does not output the electric current even if the light receiver 132 receives the reflection of the green light. The light receiver 132 includes, for example, a photodiode or a photodetector.

The converter 134 includes a current-voltage converter 1341, an amplifier 1342, and an Analog/Digital (A/D) converter 1343. The converter 134 converts the electric current received from the light receiver 132 to a digital signal and outputs the digital signal to the CPU 11.

The current-voltage converter 1341 converts the electric current received from the light receiver 132 to voltage. The current-voltage converter 1341 outputs the voltage obtained by the conversion to the amplifier 1342. The amplifier 1342 amplifies the voltage received from the current-voltage converter 1341. The amplifier 1342 outputs the amplified voltage to the A/D converter 1343. The A/D converter 1343 converts the voltage received from the amplifier 1342 to a digital signal. The digital signal obtained by the conversion is a signal indicating the intensity of the voltage received from the amplifier 1342. In other words, it can be said that the digital signal obtained by the conversion indicates the intensity of the reflection received by the light receiver 132. The A/D converter 1343 outputs the digital signal obtained by the conversion to the CPU 11. The light receiver 132 and the converter 134 are examples of the “measurement unit.”

The CPU 11 performs various types of control for the pulse wave measurement apparatus 1 according to a program stored in the storage unit 12. The CPU 11 is also referred to as a microprocessor unit (MPU) or a processor. The CPU 11 is not limited to a single processor and may have a multi-processor configuration. Moreover, the single CPU 11 connected to a single socket may have a multi-core configuration. For example, the CPU 11 controls the measurement unit 13 and the display unit 14 according to instructions received from a subject to be measured via the operation unit 15.

For example, the CPU 11 controls the controller 133 and causes the green light emitter 1312 of the light emitter 131 to emit the green light. The CPU 11 measures the intensity of the reflection of the green light based on the digital signal received from the A/D converter 1343. The CPU 11 determines whether the finger F is pressed against the light transmitting plate 135 at the suitable force, based on the intensity of the reflection of the green light from the finger F pressed against the light transmitting plate 135. When the CPU 11 determines that the finger F is pressed against the light transmitting plate 135 at the suitable force, the CPU 11 controls the controller 133 and causes the infrared light emitter 1311 of the light emitter 131 to emit the infrared light. The CPU 11 measures the pulse wave based on the intensity of the reflection of the infrared light from the finger F pressed against the light transmitting plate 135. The CPU 11 is an example of the “measurement unit” and the “execution unit.”

The storage unit 12 is described as an example of a storage unit directly accessed by the CPU 11. For example, the range of the force at which the finger is pressed against the light transmitting plate 135 suitable for the measurement of the pulse wave is stored in the storage unit 12. Moreover, correspondence relationships between the intensity of pulse and the digital signal inputted from the A/D converter 1343 to the CPU 11 are stored in the storage unit 12.

The storage unit 12 includes a Random Access Memory (RAM) and a Read Only Memory (ROM). The storage unit 12 may include external storage devices such as an Erasable Programmable ROM (EPROM), a Solid State Drive (SSD), and a Hard Disk Drive (HDD).

The display unit 14 displays various pieces of information in response to instructions from the CPU 11. For example, the display unit 14 displays information indicating whether the finger is pressed against the light transmitting plate 135 at the suitable force or not and information indicating the measured pulse wave. The display unit 14 is, for example, a Cathode Ray Tube (CRT) display, a Liquid Crystal Display (LCD), a Plasma Display Panel (PDP), an Electroluminescence (EL) panel, or an organic EL panel.

The operation unit 15 receives operations from the user of the pulse wave measurement apparatus 1. The pulse wave measurement apparatus 1 starts the measurement of the pulse wave according to an instruction of the user received via the operation unit 15. The operation unit 15 is, for example, a keyboard, a pointing device, a touch panel, or an audio input device.

The power supply unit 16 supplies power to the pulse wave measurement apparatus 1. The power supply unit 16 may be a primary cell or a secondary cell. Moreover, the pulse wave measurement apparatus 1 may receive power from a household power socket.

(Wavelength of Light and Light Reach Distance in Finger)

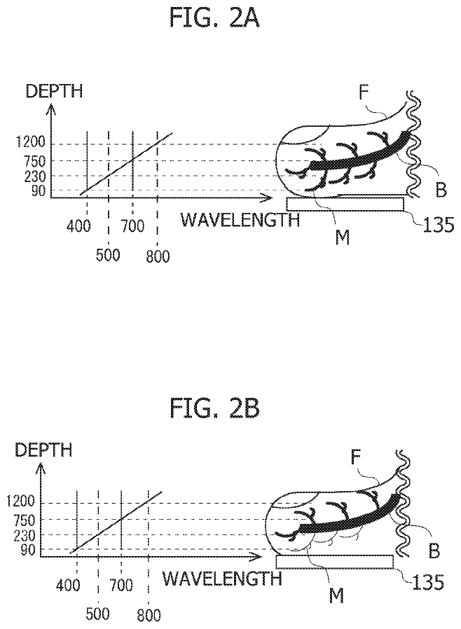

FIGS. 2A and 2B are views schematically illustrating relationships between the wavelength of light casted on the finger pressed against the light transmitting plate and depth which the casted light reaches in the finger. The vertical axes of FIGS. 2A and 2B represent the depth which the casted light reaches in the finger F (unit: μm) and the horizontal axes represent the wavelength of the light (unit: nm). As schematically illustrated in FIGS. 2A and 2B, the blood vessels in the finger F include the artery B and the capillaries M extending from the artery B toward the skin surface. Note that, in FIGS. 2A and 2B, the artery B is illustrated to represent the artery and the vein and the illustration of the vein is omitted. FIG. 2A illustrates an example in which the finger F is pressed against the light transmitting plate 135 at the force suitable for the measurement of the pulse wave and FIG. 2B illustrates an example in which the finger F is pressed against the light transmitting plate 135 at force excessively high for the measurement of the pulse wave.

With reference to FIG. 2, it can be understood that the longer the wavelength of the light is, the deeper the light can reach into the finger F. It is found that the green light with the wavelength within a range of about 490 nm to 570 nm reaches a depth of about 230 μm from the surface of the finger F. The depth of about 230 μm from the surface of the finger F is a depth reaching the capillaries M in the finger F but hardly reaching the artery B located deeper than the capillaries M. Accordingly, the intensity of the reflection received by the light receiver 132 when the green light is casted on the finger F changes depending on the flow rate of the blood flowing in the capillaries M.

It is understood from comparison between FIGS. 2A and 2B that, when the finger F is strongly pressed against the light transmitting plate 135, the capillaries M in the finger F are crushed to become narrow and the flow rate of the blood flowing into the capillaries M decreases. Moreover, the lower the force at which the finger is pressed against the light transmitting plate 135 is, the less the capillaries M are crushed and the flow rate of the blood flowing into the capillaries M thus increases. As described above, the intensity of the reflection received by the light receiver 132 when the green light is casted on the finger changes depending on the flow rate of the blood flowing in the capillaries M. Accordingly, when the finger F is strongly pressed against the light transmitting plate 135, the amount of the green light absorbed by the blood in the capillaries M decreases and the intensity of the reflection received by the light receiver 132 increases. Moreover, when the finger F is weakly pressed against the light transmitting plate 135, the amount of the green light absorbed by the blood in the capillaries M increases and the intensity of the reflection received by the light receiver 132 decreases. Specifically, the intensity of the reflection of the green light received by the light receiver 132 changes depending on the magnitude of the force at which the finger F is pressed against the light transmitting plate 135.

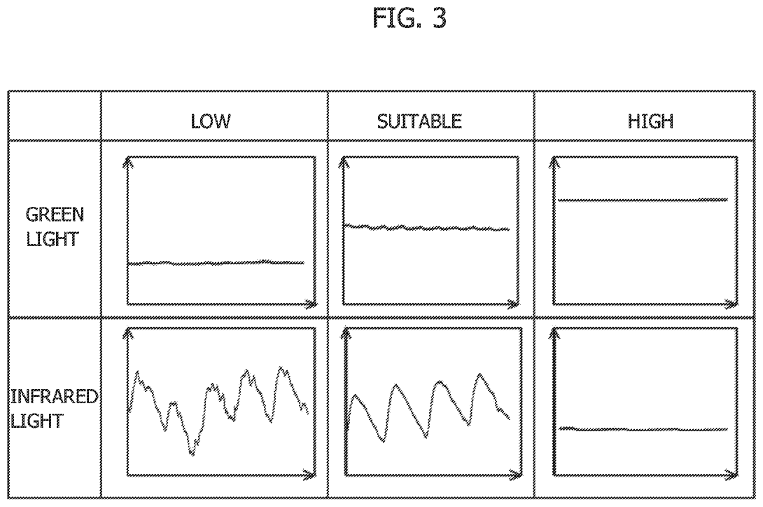

FIG. 3 is a view illustrating an example of correspondence among the magnitude of the force at which the finger is pressed against the light transmitting plate, the intensity of the reflection of the green light received by the light receiver, and the measured pulse wave in the first embodiment. In FIG. 3, graphs in the upper row illustrate examples of the intensity of the reflection and graphs in the lower row illustrate examples of the waveform of the measured pulse wave. In the graphs in the upper row, the vertical axes represent the intensity of the reflection and the horizontal axes represent time. Moreover, in the graphs in the lower row, the vertical axes represent the intensity of pulses and the horizontal axes represent time. FIG. 3 illustrates three cases where the force at which the finger F is pressed against the light transmitting plate is “low,” “suitable,” and “high” as examples.

With reference to the graphs in the upper section of FIG. 3, it can be understood that the intensity of the reflection changes substantially linearly depending on the magnitude of the force at which the finger F is pressed. In other words, it can be said that the intensity of the reflection changes substantially proportional to the magnitude of the force at which the finger F is pressed. Specifically, the higher the force at which the finger F is pressed is, the higher the intensity of the reflection is and the lower the force at which the finger F is pressed is, the lower the intensity of the reflection is.

Moreover, with reference to FIG. 3, it can be understood that, when the finger F is pressed against the light transmitting plate 135 at the suitable force, the pulse wave measurement apparatus 1 can measure the pulse wave with few noises. However, when the finger F is pressed against the light transmitting plate 135 at the low force, many noise components are included in the measured pulse wave (clean waveform is not obtained). Moreover, when the finger F is pressed against the light transmitting plate 135 at the high force, no waveform of the pulse wave can be obtained. Accordingly, in the embodiment, the pulse wave measurement apparatus 1 determines whether the finger F is pressed against the light transmitting plate 135 at suitable force or not based on the intensity of the reflection of the green light received by the light receiver 132 and, when determining that the finger F is pressed at the suitable force, measures the pulse wave. Note that the range of the intensity of the reflection in which the pulse wave measurement apparatus 1 determines that the finger F is pressed against the light transmitting plate 135 at the suitable force may be determined through experiments or the like and stored in the storage unit 12.

(Timing Chart)

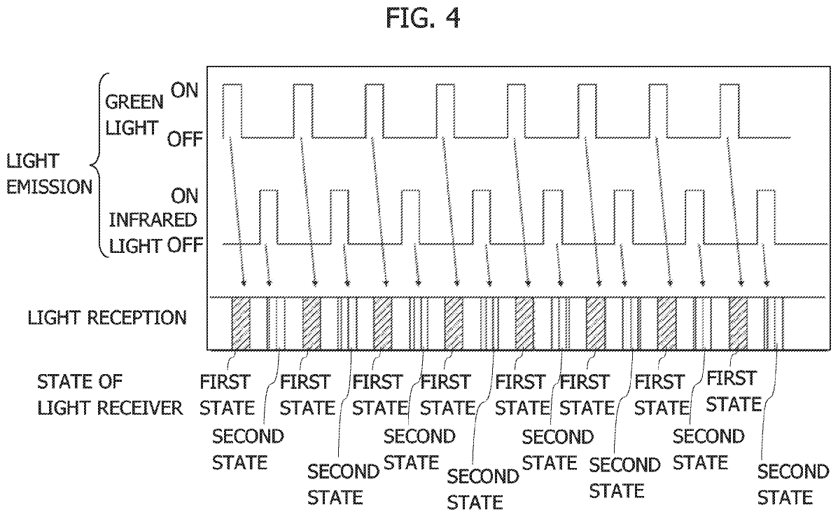

FIG. 4 is an example of the timing chart of the pulse wave measurement apparatus according to the first embodiment. In the timing chart of FIG. 4, charts in the upper two rows illustrate examples of timings at which the infrared light emitter 1311 and the green light emitter 1312 emit light. Moreover, a chart in the bottom row illustrates an example of periods in which the light receiver 132 is in the first state and periods in which the light receiver 132 is in the second state.

In the charts in the upper two rows of FIG. 4, “ON” indicates periods in which the light emitter 131 emits light and “OFF” indicates periods in which the light emitter 131 is not emitting light (turned off). Specifically, in FIG. 4, the periods in which the green light is “ON” indicate periods in which the green light emitter 1312 is emitting light and the periods in which the green light is “OFF” indicate periods in which the green light emitter 1312 is not emitting light. Moreover, in FIG. 4, the periods in which the infrared light is “ON” indicate periods in which the infrared light emitter 1311 is emitting light and the periods in which the infrared light is “OFF” indicate periods in which the infrared light emitter 1311 is not emitting light.

In the chart in the bottom row of FIG. 4, rectangles described as the first state (rectangles hatched with oblique lines in FIG. 4) illustrate an example of a state where the light receiver 132 can receive the green light and generate the electric current. Moreover, rectangles described as the second state (rectangles hatched by vertical lines in FIG. 4) illustrate an example of a state where the light receiver 132 can receive the infrared light and generate the electric current.

With reference to FIG. 4, it can be understood that the light receiver 132 is set to the first state after the light emitter 131 emits the green light and the first state is terminated before the light emitter 131 emits the infrared light. Moreover, the light receiver 132 is set to the second state after the light emitter 131 emits the infrared light and the second state is terminated before the light emitter 131 emits the green light. Moreover, the light receiver 132 is set to an off state between the first state and the second state. The off state is a state where the light receiver 132 generates no electric current even if the light receiver 132 receives light and is, for example, a state where the power supply unit 16 stops supplying power to the light receiver 132.

(Processing Flow)

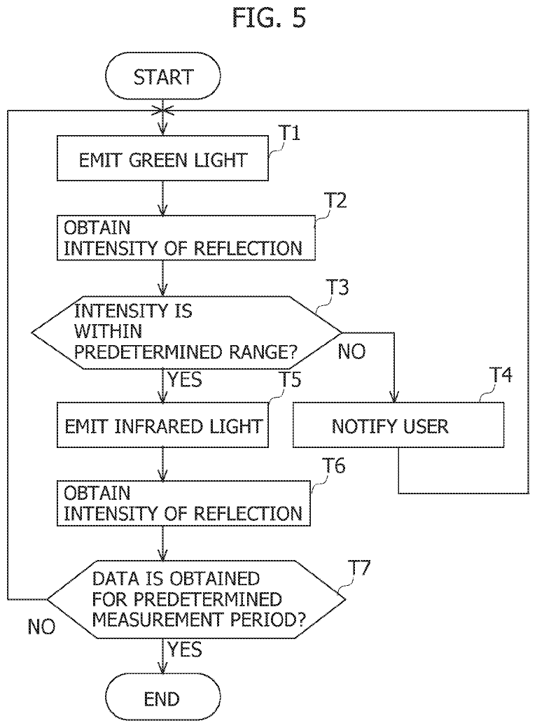

FIG. 5 is a view illustrating an example of a processing flow of the pulse wave measurement apparatus according to the first embodiment. For example, the processing flow illustrated in FIG. 5 starts when the user gives an instruction to start the measurement of the pulse wave on the operation unit 15 while pressing the finger F against the light transmitting plate 135. An example of the processing flow of the pulse wave measurement apparatus according to the first embodiment is described below with reference to FIG. 5.

In the processing from T1 to T5, whether the finger is pressed against the light transmitting plate 135 at the force suitable for the measurement of the pulse wave or not is determined. At T1, the CPU 11 instructs the controller 133 to emit the green light. The controller 133 controls the light emitter 131 such that the green light emitter 1312 in the light emitter 131 emits light. The green light emitter 1312 performs light emission to emit the green light.

At T2, the green light emitted at T1 is casted on the finger F though the light transmitting plate 135. The green light casted on the finger F is partially absorbed by the blood flowing in the capillaries in the finger F. Part of the green light not absorbed is reflected on the finger F and enters the light receiver 132. The light receiver 132 switches between the first state and the second state in a time division manner as described above. At timing T2 after the emission of the green light at T1, the light receiver 132 is set to the first state and outputs the electric current depending on the reception intensity of the reflection of the green light.

The electric current outputted by the light receiver 132 is converted to voltage by the current-voltage converter 1341. The voltage obtained in the conversion by the current-voltage converter 1341 is amplified by the amplifier 1342 and is inputted into the A/D converter 1343. The A/D converter 1343 converts the voltage received from the amplifier 1342 to the digital signal indicating the reception intensity of the reflection of the green light and inputs the digital signal obtained by the conversion into the CPU 11. The CPU 11 obtains the reception intensity of the reflection of the green light received by the light receiver 132 by obtaining the digital signal from the A/D converter 1343. The CPU 11 determines whether the obtained reception intensity of the reflection of the green light is within the predetermined range which indicates that the finger F is pressed at the force suitable for the measurement of the pulse wave. When the reception intensity of the reflection of the green light is within the predetermined range (YES in T3), the processing proceeds to T5. When the reception intensity of the reflection of the green light is outside the predetermined range (NO in T3), the processing proceeds to T4.

At T4, the CPU 11 causes the display unit 14 to output a message indicating that the finger is not pressed against the light transmitting plate 135 at the suitable force. For example, when the CPU 11 determines that the finger is pressed at excessively high force at T3, the CPU 11 causes the display unit 14 to output a message indicating that the finger is pressed at excessively high force. Moreover, for example, when the CPU 11 determines that the finger is pressed at excessively low force in T3, the CPU 11 causes the display unit 14 to output a message indicating that the finger is pressed at excessively low force.

In the processing from T5 to T7, the measurement of the pulse wave is executed. At T5, the CPU 11 instructs the controller 133 to emit the infrared light. The controller 133 controls the light emitter 131 such that the infrared light emitter 1311 of the light emitter 131 emits light. The infrared light emitter 1311 performs light emission to emit the infrared light.

At T6, the infrared light emitted at T5 passes through the light transmitting plate 135 and is casted on the finger F. The infrared light casted on the finger F is partially absorbed by the blood flowing in the capillaries and the artery in the finger. Part of the infrared light not absorbed is reflected on the finger F and enters the light receiver 132. At timing T6 after the emission of the infrared light at T5, the light receiver 132 is in the second state and outputs the electric current depending on the reception intensity of the reflection of the infrared light.

The electric current outputted by the light receiver 132 is inputted into the CPU 11 via the current-voltage converter 1341, the amplifier 1342, and the A/D converter 1343 as the digital signal indicating the reception intensity of the reflection of the infrared light. The CPU 11 obtains the reception intensity of the reflection of the infrared light received by the light receiver 132 by obtaining the digital signal from the A/D converter 1343. For example, the CPU 11 measures the pulse wave based on the correspondence relationships between the digital signal and the intensity of the pulse stored in the storage unit 12 and stores data indicating the measured pulse wave in the storage unit 12.

When the data of the pulse wave is obtained for a predetermined measurement period (YES in T7), the processing is terminated. When the data of the pulse wave is not obtained for the predetermined measurement period (NO in T7), the processing returns to T1. In the first embodiment, the processing from T1 to T7 is repeated at an interval of 0.5 seconds and the measurement of the pulse wave is performed when the force at which the finger F is pressed against the light transmitting plate 135 is within the range suitable for the measurement of the pulse wave, and is not performed when the force at which the finger F is pressed against the light transmitting plate 135 is outside the range suitable for the measurement of the pulse wave. The pulse wave measurement apparatus 1 according to the first embodiment can measure the pulse wave more accurately by performing such processing.

### Comparative Example

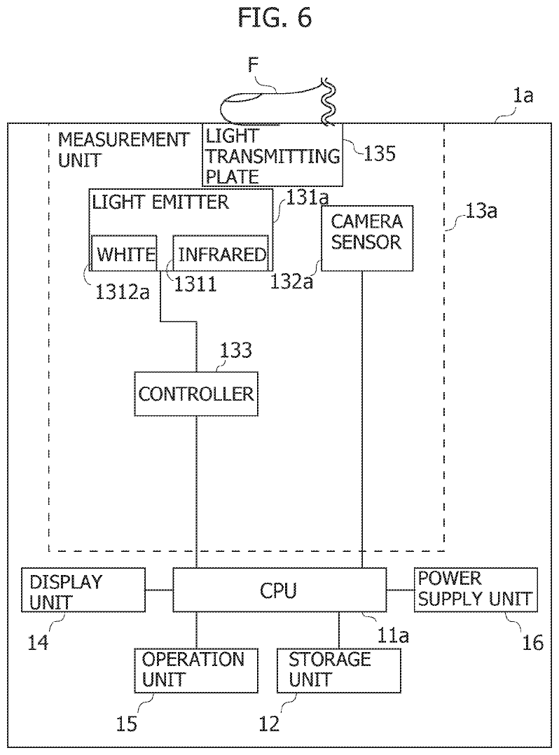

A comparative example is described to study the effects of the embodiment. FIG. 6 is a view illustrating an example of a pulse wave measurement apparatus according to the comparative example. The pulse wave measurement apparatus 1a according to the comparative example employs white light to determine whether the finger is pressed against the light transmitting plate at the suitable force or not, unlike the pulse wave measurement apparatus 1 according to the first embodiment. In FIG. 6, the same components as those in FIG. 1 are denoted by the same reference numerals and description thereof is omitted.

The pulse wave measurement apparatus 1a according to the comparative example is different from the pulse wave measurement apparatus 1 according to the first embodiment in that the pulse wave measurement apparatus 1a includes a measurement unit 13a. The measurement unit 13a includes a light emitter 131a, a camera sensor 132a, and the controller 133.

The light emitter 131a is different from the light emitter 131 according to the first embodiment in that the light emitter 131a includes a white light emitter 1312a configured to emit white light toward the light transmitting plate 135, instead of the green light emitter 1312 configured to emit the green light. The light emitter 131a emits the white light or the infrared light toward the light transmitting plate 135 by emitting light in response to an instruction from the controller 133. The finger F pressed against the light transmitting plate 135 is irradiated with the light emitted from the light emitter 131a.

The camera sensor 132a is, for example, a digital camera including a Charge Coupled Device (CCD) sensor or a Complementary Metal Oxide Semiconductor (CMOS) sensor. The camera sensor 132a captures an image of the finger F pressed against the light transmitting plate 135 with the finger irradiated with the light from the light emitter 131a and generates image data.

For example, a CPU 11a controls the controller 133 and causes the white light emitter 1312a of the light emitter 131a to emit the white light. The CPU 11a obtains the image data of the finger irradiated with the white light from the camera sensor 132a. The CPU 11a determines whether the finger is pressed against the light transmitting plate 135 at the suitable force or not based on the color of the finger in the obtained image data.

When the CPU 11a determines that the finger F is pressed against the light transmitting plate 135 at the suitable force, the CPU 11a controls the controller 133 and causes the infrared light emitter 1311 of the light emitter 131 to emit the infrared light. The CPU 11a measures the pulse wave based on the intensity of the reflection of the infrared light from the finger F pressed against the light transmitting plate 135.

In the comparative example, as described above, the white light is employed to determine whether the finger F is pressed against the light transmitting plate 135 at the suitable force or not. The white light includes light of wavelengths in a broad wavelength band of about 400 nm to 700 nm. With reference to FIG. 2, it can be understood that, when light of a wavelength around 700 nm is casted on the finger F, the casted light reaches the artery B in the finger F. Accordingly, when the camera sensor 132a captures the image of the finger F with the white light casted on the finger, the color of the finger F in the captured image data tends to be reddish.

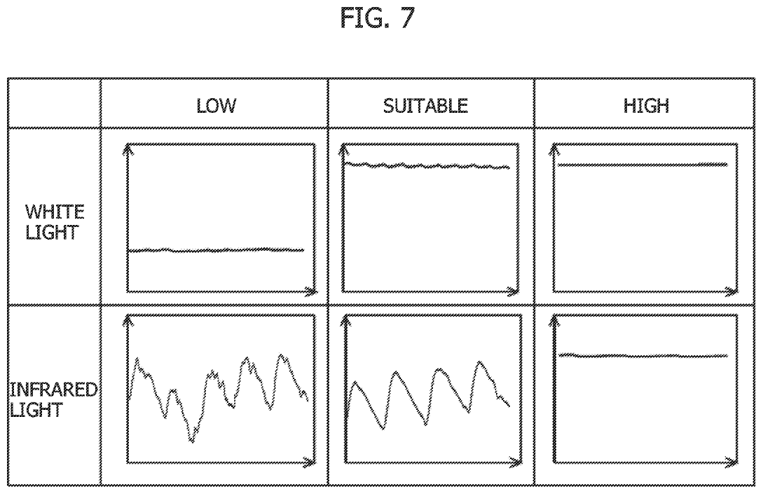

FIG. 7 is a view illustrating an example of correspondence among the magnitude of the force at which the finger is pressed against the light transmitting plate, the degree of redness in the color of the finger captured by the camera sensor, and the measured pulse wave in the comparative example. In FIG. 7, graphs in the upper row illustrate examples of the degree of redness in the color of the finger F captured by the camera sensor and graphs in the lower row illustrate the waveform of the measured pulse wave. In the graphs in the upper row, the vertical axes represent the degree of redness and the horizontal axes represent time. Moreover, in the graphs in the lower row, the vertical axes represent the intensity of pulses and the horizontal axes represent time. FIG. 7 illustrates three cases where the force at which the finger F is pressed against the light transmitting plate is “low,” “suitable,” and “high” as examples.

With reference to FIG. 7, it can be understood that there is no large difference in the degree of redness between the case where the finger F is pressed against the light transmitting plate 135 at the suitable force and the case where the finger F is strongly pressed against the light transmitting plate 135. Accordingly, it is difficult for the pulse wave measurement apparatus is to distinguish the case where the color of the finger is red due to the finger F being pressed against the light transmitting plate 135 at excessively high force and the case where the color of the finger is red due to irradiation of the artery B in the finger, though the finger F is pressed against the light transmitting plate 135 at the suitable force.

Moreover, when the skin color of the user is dark (when the user has a dark skin or the user has a dark skin due to suntan or the like), the color of the skin F changes little even if the force at which the finger F is pressed against the light transmitting plate 135 changes. Accordingly, it is difficult for the pulse wave measurement apparatus 1a to determine whether the finger F is pressed against the light transmitting plate 135 at the suitable force or not when the skin color of the user is dark.

Meanwhile, in the pulse wave measurement apparatus 1 according to the first embodiment, the intensity of the reflection of the green light casted on the finger F is used to determine whether the finger F is pressed against the light transmitting plate 135 at the suitable force or not. As described above, the green light casted on the finger F reaches capillaries M in the finger F but hardly reaches the artery B located deeper than the capillaries M. Thus, the blood flowing in the capillaries M affects the intensity of reflection more than the blood flowing in the artery B. As described above, the flow rate of the blood in the capillaries M changes depending on the force at which the finger F is pressed against the light transmitting plate 135. Accordingly, the pulse wave measurement apparatus 1 according to the first embodiment determines whether the finger F is pressed against the light transmitting plate 135 at the suitable force or not based on the intensity of the reflection of the green light casted on the finger F and can thereby perform the determination more accurately than the pulse wave measurement apparatus 1a according to the comparative example.

Moreover, the pulse wave measurement apparatus 1 determines whether the finger F is pressed against the light transmitting plate 135 at the suitable force or not based on the intensity of reflection instead of the color of the finger F. Accordingly, the pulse wave measurement apparatus 1 according to the first embodiment can determine whether the finger is pressed at the suitable force or not more accurately than the comparative example even if the skin color of the finger F is dark.

Various changes can be made to the embodiment disclosed above as long as there is no technical contradiction.

<<Computer Readable Recording Medium>>

An information processing program which causes a computer or any other machine or apparatus (hereafter, referred to as computer or the like) to implement any of the aforementioned functions can be recorded in a recording medium readable by the computer or the like. The functions can be provided by causing the computer or the like to read and execute the program in the recording medium.

The recording medium readable by the computer or the like refers to a recording medium which stores information such as data and the program by means of an electrical, magnetic, optical, mechanical, or chemical action and from which the information can be read by using the computer or the like. Recording media removable from the computer or the like among such recording media include, for example, a flexible disk, a magneto-optical disc, a Compact Disc Read Only Memory (CD-ROM), a Compact Disc-Recordable (CD-R), a Compact Disc-ReWriterable (CD-RW), Digital Versatile Disc (DVD), a Blu-ray Disc (BD), a Digital Audio Tape (DAT), an 8-mm tape, a memory card such as a flash memory, and the like. Moreover, recording media fixed to the computer or the like include a hard disk drive, a ROM, and the like.

The pulse wave measurement apparatus can more accurately determine that the finger is pressed at suitable force.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A pulse wave measurement apparatus comprising:
a light transmitting plate which is made of a member capable of transmitting light and against which a finger is pressed;
a green light which casts green light;
an infrared light which casts infrared light;
a memory; and
a processor coupled to the memory and the processor configured to:
cause the green light to cast green light on the finger through the light transmitting plate, the green light having a peak wavelength in a wavelength range of 490 nm or more and 570 nm or less;
detect an intensity of reflection of the casted green light from the finger;
measure a pulse wave in the finger by making the infrared light cast infrared light on the finger through the light transmitting plate when the intensity of the reflection indicates that force at which the finger is pressed against the light transmitting plate is within a predetermined range suitable for the measurement of the pulse wave.

2. The pulse wave measurement apparatus according to claim 1, wherein the processor transitions to a state in which the processor is capable of detecting the intensity of the reflection at least in part of a period from a moment when the green light is emitted to a moment when the infrared light is emitted.

3. The pulse wave measurement apparatus according to claim 1, wherein the processor cancels the casting of the infrared light by the infrared light when the intensity of the reflection indicates that the force is outside the predetermined range.

4. The pulse wave measurement apparatus according to claim 1, wherein the processor notifies a user that the force at which the finger is pressed against the light transmitting plate is outside the predetermined range when the intensity of the reflection indicates that the force is outside the predetermined range.
